(19) United States
(12) Patent Application Publication
(10) Pub. No.: US 2020/0337805 A1
(43) Pub. Date: Oct. 29, 2020

(54) Monitoring Apparatus for Wearing of Dental Braces and Monitoring Method of the Same

(71) Applicant: Yi-Ju Su, Minxiong Township (TW)

(72) Inventor: Yi-Ju Su, Minxiong Township (TW)

(21) Appl. No.: 16/396,810

(22) Filed: Apr. 29, 2019

(51) Int. Cl. A61C 7/08; A61C 7/10; A61B 5/00; A61C 7/36

(52) U.S. Cl. CPC A61C 7/08; A61C 7/10; A61C 7/002; A61C 7/36; A61B 5/682

(57) ABSTRACT

A monitoring apparatus for wearing of dental braces executed with dental braces has a signal component, a container, and a reader. The signal component is disposed on the dental braces. The container is used to store the dental braces and has a power supply unit. The reader is disposed in the container, is electrically connected to the power supply unit, is capable of reading a signal from the signal component, and has a reading unit, a central processing unit, a memory unit, and a mercury battery. The reading unit reads the signal from the signal component. The central processing unit is electrically connected to the reading unit. The memory unit is electrically connected to the central processing unit. The mercury battery is electrically connected to the central processing unit. A monitoring method of a monitoring apparatus for wearing of dental braces is also provided.

### BACKGROUND OF THE INVENTION

### 1. Field of the Invention

The present invention relates to a monitoring apparatus for wearing of dental braces and a monitoring method of the same, and more particularly to a monitoring apparatus for wearing of dental braces that enables dentists to track on progress of a treatment, and to make adjustment to the treatment accordingly.

### 2. Description of Related Art

After an orthodontic treatment comes to an end, dentists remove braces for patients and provide the patients with removable dental braces. Patients are asked to wear the removable dental braces practically all the time except when eating or brushing teeth to avoid further malposition of teeth. However, patients often forget to put on the removable dental braces after they remove the removable dental braces. In addition to that, some patients do not follow instructions of the dentists considering inconvenience or appearance. The above-mentioned conditions all lead to unnecessary extended treatment and new displacements of teeth. If the new displacements of teeth are too serious, the treatment may have to be restarted. Even if the patients return for checkup on a regular basis, the dentists cannot track on progress of the orthodontic treatment. To overcome the shortcoming, the present invention provides a monitoring apparatus for wearing of dental braces and a monitoring method of the same to mitigate or obviate the aforementioned problems.

### SUMMARY OF THE INVENTION

The main objective of the present invention is to provide a monitoring apparatus for wearing of dental braces and a monitoring method of the same.

The monitoring apparatus for wearing of dental braces executed with dental braces comprises a signal component, a container, and a reader. The signal component is disposed on the dental braces. The container is used to store the dental braces disposed with the signal component and has a power supply unit disposed therein. The reader is disposed in the container, is electrically connected to the power supply unit, is capable of reading a signal emitted from the signal component, and has a reading unit, a central processing unit, a memory unit, and a mercury battery. The reading unit is capable of reading the signal emitted from the signal component. The central processing unit is electrically connected to the reading unit. The memory unit is electrically connected to the central processing unit. The mercury battery is electrically connected to the central processing unit.

The monitoring method of the monitoring apparatus for wearing of dental braces is executed with dental braces and comprises steps as follows: a preparing step, a first reading step, a processing step, a storing step, a converting step, and a second reading step. The preparing step comprises preparing the above-mentioned monitoring apparatus for wearing of dental braces. In the first reading step, the reading unit of the reader reads a signal emitted from the signal component. In the processing step, the central processing unit of the reader processes the signal read by the reading unit. In the storing step, the memory unit of the reader stores the signal processed by the central processing unit. In the converting step, the central processing unit of the reader converts the signal stored by the memory unit to a time signal and attains a stored value. In the second reading step, the stored value attained in the converting step is read.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view showing a signal component of a monitoring apparatus for wearing of dental braces disposed on dental braces in accordance with the present invention;

FIG. 2 is a perspective view showing the dental braces stored in a container of the monitoring apparatus for wearing of dental braces in FIG. 1;

FIG. 3 is a circuit block diagram of the monitoring apparatus for wearing of dental braces in FIG. 2; and

FIG. 4 is a flow diagram of a monitoring method of the monitoring apparatus for wearing of the dental braces in accordance with the present invention.

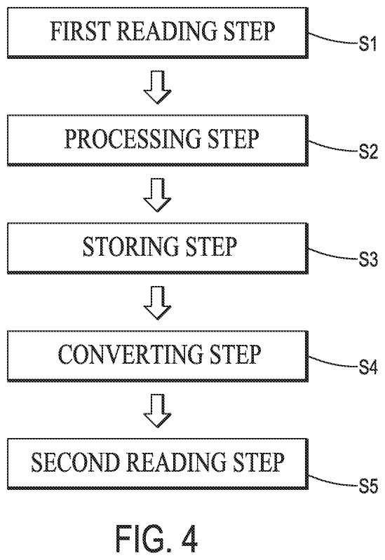

### DETAILED DESCRIPTION OF THE PREFERRED EMBODIMENT

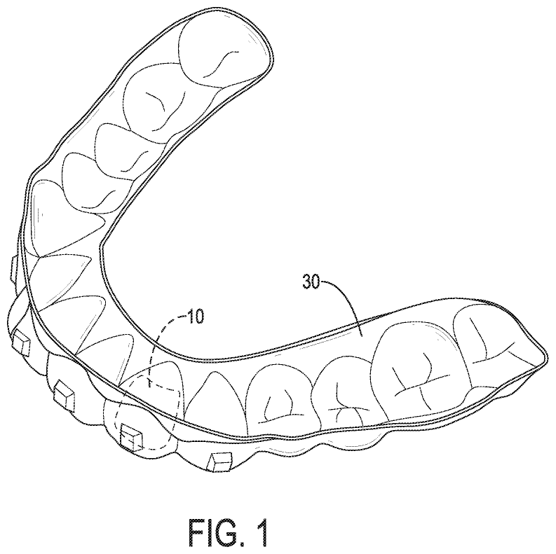

With reference to FIGS. 1 to 3, a monitoring apparatus for wearing of dental braces in accordance with the present invention comprises a signal component 10, a container 40, and a reader 20. The signal component 10 is disposed on dental braces 30. Furthermore, the signal component 10 is bonded to the dental braces 30 by resin. The container 40 is used to store the dental braces 30. The reader 20 is disposed in the container 40, and the reader 20 is capable of reading a signal emitted from the signal component 10.

With reference to FIG. 1, in the present invention, the signal component 10 is a RFID (Radio Frequency Identification; RFID) chip, and the signal component 10 serves as a signal source. Moreover, a UHF (Ultra High Frequency) RFID chip can be the signal component 10, and is capable of detecting temperature and humidity of the environment.

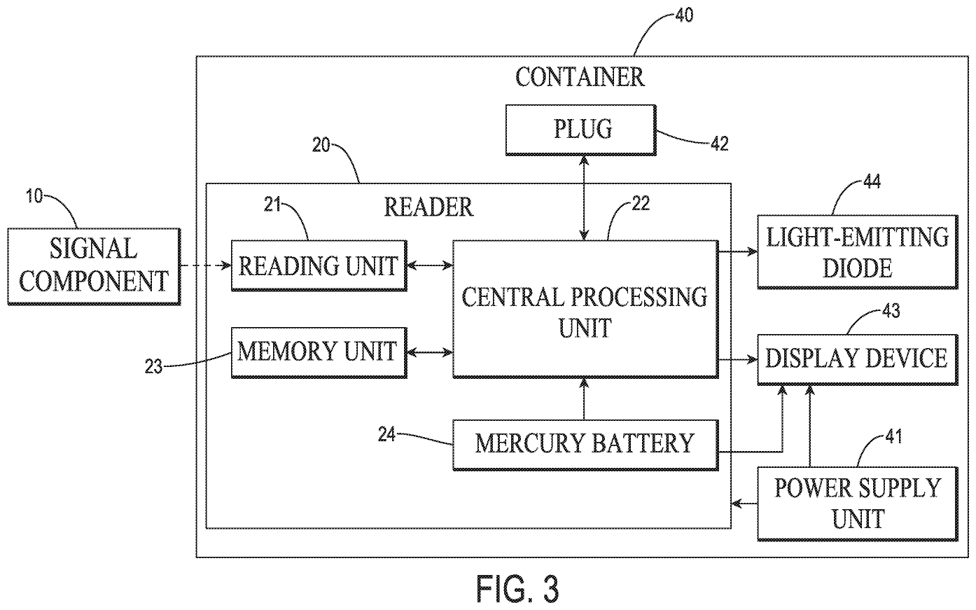

With reference to FIG. 3, the reader 20 is capable of reading the signal emitted from the signal component 10 and comprises a reading unit 21, a central processing unit 22, a memory unit 23, a mercury battery 24, and a circuit board. The reading unit 21 is capable of reading the signal emitted from the signal component 10. The central processing unit 22 is electrically connected to the reading unit 21. The memory unit 23 is electrically connected to the central processing unit 22 to store signals processed by the central processing unit 22. In addition, the memory unit 23 may be a memory. The mercury battery 24 is electrically connected to the central processing unit 22 to supply electricity to the central processing unit 22. The reading unit 21, the central processing unit 22, the memory unit 23, and the mercury battery 24 are disposed on the circuit board. Besides, the circuit board is a printed circuit board and is disposed in the container 40.

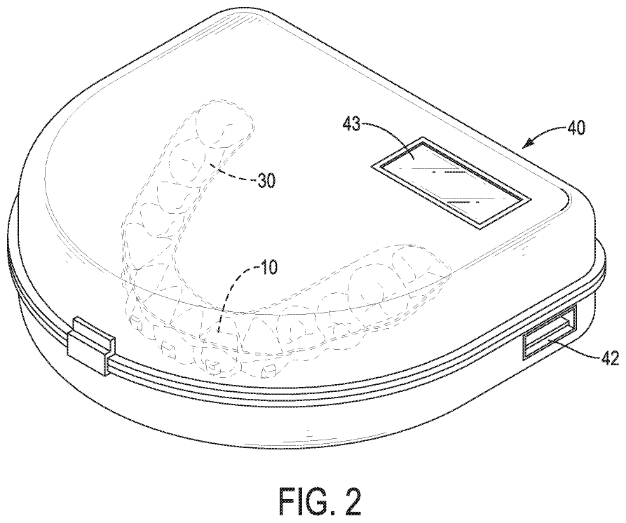

With reference to FIGS. 2 and 3, the container 40 comprises a power supply unit 41, a plug 42, a display device 43, and a light-emitting diode 44. The power supply unit 41 is disposed in the container 40, and is electrically connected to the reader 20 to supply the reader 20 with electricity. Besides, the power supply unit 41 is a lithium battery. The plug 42 is disposed on the container 40 and is electrically connected to the central processing unit 22 of the reader 20. In the present invention, the plug 42 is a universal serial bus (USB) plug. The display device 43 is disposed on the container 40, and is electrically connected to the central processing unit 22 of the reader 20, the power supply unit 41, and the mercury battery 24 of the reader 20. The light-emitting diode 44 is disposed on the container 40, and is electrically connected to the central processing unit 22 of the reader 20. The central processing unit 22 has a threshold value. When the central processing unit 22 detects that a battery level of the power supply unit 41 is lower than that of the threshold value of the central processing unit 22, the light-emitting diode 44 glows to remind users that the power supply unit 41 needs to be replaced. At the same time, power supplier of the reader 20 and the display device 43 changes from the power supply unit 41 to the mercury battery 24. When the battery level of the power supply unit 41 is higher than that of the threshold value of the central processing unit 22, the power supply unit 41 supplies the reader 20 and the display device 43 with electricity.

Furthermore, a monitoring method of the monitoring apparatus for wearing of the dental braces executed by the monitoring apparatus for wearing of dental braces is also provided in the present invention. The monitoring apparatus for wearing of dental braces starts working as soon as the dental braces 30 are stored in the container 40. The monitoring method of the monitoring apparatus for wearing of the dental braces comprises the following steps:

S1. A first reading step: The reading unit 21 of the reader 20 reads a signal emitted from the signal component 10.

S2. A processing step: The central processing unit 22 of the reader 20 processes the signal read by the reading unit 21.

S3. A storing step: The memory unit 23 of the reader 20 stores the signal processed by the central processing unit 22.

S4. A converting step: The central processing unit 22 of the reader 20 converts the signal stored by the memory unit 23 to a time signal. A stored value of the time during which the dental braces 30 are stored in the container 40 can be therefore attained from the time signal.

S5. A second reading step: The stored value can be read by one of the two methods:

A. Read the stored value via the display device 43: The display device 43 reads and displays the stored value. Dentists can learn a cumulative time during which patients did not wear the dental braces 30 from the display device 43.

B. Read the stored value via a peripheral device: Connect the plug 42 of the container 40 to a computer installed with a reading software. The reading software reads the stored value from the plug 42 and displays the stored value. Dentists can learn the cumulative time during which patients did not wear the dental braces 30 from the reading software.

When patients return for checkup, dentists can read the stored value displayed on the display device 43. Also, dentists can connect the plug 42 of the container 40 to the computer installed with the reading software and read the stored value from the reading software. Knowing the stored value, dentists can attain the cumulative time during which the dental braces 30 are stored in the container 40. The cumulative time during which the patients did not wear the dental braces 30 can be therefore attained. Overall, the monitoring apparatus for wearing of dental braces and the monitoring method of the same enable dentists to track on progress of a treatment, and make adjustment to the treatment accordingly.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A monitoring method of a monitoring apparatus for wearing of dental braces, the method adapted to be executed in cooperation with dental braces and comprising steps of:
a preparing step comprising preparing a monitoring apparatus for wearing of dental braces, and the monitoring apparatus having
a signal component disposed on the dental braces;
a container being used to store the dental braces disposed with the signal component and having
a power supply unit disposed therein; and
a reader disposed in the container, electrically connected to the power supply unit, being capable of reading a signal emitted from the signal component, and having
a reading unit being capable of reading the signal emitted from the signal component;
a central processing unit electrically connected to the reading unit;
a memory unit electrically connected to the central processing unit; and
a mercury battery electrically connected to the central processing unit;
a first reading step: the reading unit of the reader reading the signal emitted from the signal component;
a processing step: the central processing unit of the reader processing the signal read by the reading unit;
a storing step: the memory unit of the reader storing the signal processed by the central processing unit;
a converting step: the central processing unit of the reader converting the signal stored by the memory unit to a time signal and attaining a stored value; and
a second reading step: reading the stored value attained in the converting step.

2. The monitoring method of a monitoring apparatus for wearing of dental braces as claimed in claim 1, wherein the preparing step comprises preparing a RFID chip served as the signal component.

3. The monitoring method of a monitoring apparatus for wearing of dental braces as claimed in claim 1, wherein the preparing step comprises preparing a UHF RFID chip served as the signal component.

4. The monitoring method of a monitoring apparatus for wearing of dental braces as claimed in claim 1, wherein
in the preparing step, the container has a plug disposed on the container and electrically connected to the central processing unit of the reader; and
in the second reading step, the plug of the container is connected to a computer installed with a reading software, and the reading software reads the stored value from the plug.

5. The monitoring method of a monitoring apparatus for wearing of dental braces as claimed in claim 2, wherein
in the preparing step, the container has a plug disposed on the container and electrically connected to the central processing unit of the reader; and
in the second reading step, the plug of the container is connected to a computer installed with a reading software, and the reading software reads the stored value from the plug.

6. The monitoring method of a monitoring apparatus for wearing of dental braces as claimed in claim 3, wherein
in the preparing step, the container has a plug disposed on the container and electrically connected to the central processing unit of the reader; and
in the second reading step, the plug of the container is connected to a computer installed with a reading software, and the reading software reads the stored value from the plug.

7. The monitoring method of a monitoring apparatus for wearing of dental braces as claimed in claim 1, wherein
in the preparing step, the container has a display device disposed on the container and electrically connected to the central processing unit of the reader; and
in the second reading step, the display device reads the stored value.

8. The monitoring method of a monitoring apparatus for wearing of dental braces as claimed in claim 2, wherein
in the preparing step, the container has a display device disposed on the container and electrically connected to the central processing unit of the reader; and
in the second reading step, the display device reads the stored value.

9. The monitoring method of a monitoring apparatus for wearing of dental braces as claimed in claim 3, wherein
in the preparing step, the container has a display device disposed on the container and electrically connected to the central processing unit of the reader; and
in the second reading step, the display device reads the stored value.

10. The monitoring method of a monitoring apparatus for wearing of dental braces as claimed in claim 1, wherein
in the preparing step, the container has
a plug disposed on the container and electrically connected to the central processing unit of the reader; and
a display device disposed on the container and electrically connected to the central processing unit of the reader; and
in the second reading step, reading the stored value is executed in either way as follows:
connecting the plug of the container to a computer installed with a reading software, and the reading software reading the stored value from the plug; and
the display device reading the stored value.

11. The monitoring method of a monitoring apparatus for wearing of dental braces as claimed in claim 2, wherein
in the preparing step, the container has
a plug disposed on the container and electrically connected to the central processing unit of the reader; and
a display device disposed on the container and electrically connected to the central processing unit of the reader; and
in the second reading step, reading the stored value is executed in either way as follows:
connecting the plug of the container to a computer installed with a reading software, and the reading software reading the stored value from the plug; and
the display device reading the stored value.

12. The monitoring method of a monitoring apparatus for wearing of dental braces as claimed in claim 3, wherein
in the preparing step, the container has
a plug disposed on the container and electrically connected to the central processing unit of the reader; and
a display device disposed on the container and electrically connected to the central processing unit of the reader; and
in the second reading step, reading the stored value is executed in either way as follows:
connecting the plug of the container to a computer installed with a reading software, and the reading software reading the stored value from the plug; and
the display device reading the stored value.

13. A monitoring apparatus for wearing of dental braces executed with dental braces comprising:
a signal component disposed on the dental braces;
a container being used to store the dental braces disposed with the signal component and having
a power supply unit disposed therein; and
a reader disposed in the container, electrically connected to the power supply unit, being capable of reading a signal emitted from the signal component, and having
a reading unit being capable of reading the signal emitted from the signal component;
a central processing unit electrically connected to the reading unit;
a memory unit electrically connected to the central processing unit; and
a mercury battery electrically connected to the central processing unit.

14. The monitoring apparatus for wearing of dental braces as claimed in claim 13, wherein the signal component is a RFID chip.

15. The monitoring apparatus for wearing of dental braces as claimed in claim 13, wherein the signal component is a UHF RFID chip.

16. The monitoring apparatus for wearing of dental braces as claimed in claim 13, wherein the container has a plug disposed on the container and electrically connected to the central processing unit of the reader.

17. The monitoring apparatus for wearing of dental braces as claimed in claim 14, wherein the container has a plug disposed on the container and electrically connected to the central processing unit of the reader.

18. The monitoring apparatus for wearing of dental braces as claimed in claim 15, wherein the container has a plug disposed on the container and electrically connected to the central processing unit of the reader.
